(19) United States
(12) Patent Application Publication
(10) Pub. No.: US 2009/0239894 A1
(43) Pub. Date: Sep. 24, 2009

(54) Stabilized aqueous solutions of ergoline compounds

(76) Inventors: Bjorn Schurad, Munich (DE); Harald Mottl, Appenzell (CH)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/077,698

(22) Filed: Mar. 20, 2008

(51) Int. Cl. A61K 31/438; C07D 221/18; A61P 25/28

(52) U.S. Cl. 514/284; 546/70

(57) ABSTRACT

The present invention relates to stabilized aqueous solutions of an ergoline compound of formula I

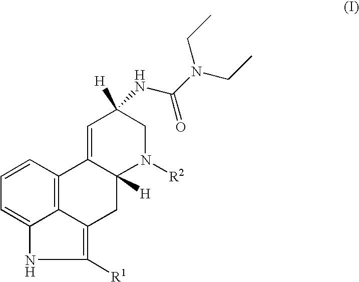

or their physiologically tolerable salt or derivative, in which R¹ stands for an H atom or a halogen atom and R² stands for an alkyl group or alkenyl group with 1 to 4 carbons and a single or double bond, also containing 0.05% to 90.00% of at least one oxygen-containing cosolvent. Likewise, the present invention relates to the use of the solutions stabilized according to the present invention to prepare an agent for parenteral treatment of neurodegenerative diseases or brain trauma.

The present invention relates to stabilized aqueous solutions of ergoline compounds or their physiologically tolerable salts or derivatives in which the aqueous solution contains 0.05 to 90.00% of at least one oxygen-containing cosolvent. The present invention likewise relates to the use of an aqueous solution stabilized in this way for production of an agent for parenteral treatment of neurodegenerative diseases or brain trauma.

Ergoline compounds such as lisuride or its physiological tolerable salts are used today for treatment of patients suffering from Parkinson's disease (F. Stocchi et al. (2002) Brain 125: 2058-2066), Meige syndrome (G. Ransmayr et al. (1988) Clinical Neuropharmacology 11: 68-76), Dystonias (N. P. Quinn et al. (1984) Neurology 34: 223) or other serious diseases. The required active ingredient solutions are administered intravenously, intramuscularly, transdermally or subcutaneously.

However, many ergoline compounds have proven to be unstable in dissolved form (water as the solvent). Especially when there are 8α-amino groups on the ring system, e.g., a diethylurea group in the case of lisuride, these compounds are extremely unstable in solution and tend to decompose rapidly (hydrolysis). Another disadvantage is the poor solubility of natural ergot alkaloids as well as their salts and derivatives in water as the solvent.

Therefore, the corresponding ergoline compounds, in particular lisuride, are currently being provided only in lyophilized form. These must be reconstituted, i.e., returned to a liquid form by the patient, by adding a corresponding salt solution before being administered to the patient.

Only after this reconstitution can the substance be administered to the patient. In particular, when using a programmable minipump or micropump, the solution must first be transferred to a special syringe. Only then can the final filling of the minipump or micropump take place.

It is therefore easy to see that the type and number of steps to be performed when using a lyophilizate make this procedure extremely complex and difficult. This procedure has proven to be extremely difficult especially for patients who suffer from movement disorders (as in Parkinson's disease) or dystonia because of their primary illness.

The lisuride solution reconstituted from the lyophilizate currently has only a limited stability so that administration of this solution by a minipump or micropump, for example, over a long period of time must be ruled out.

In addition, the multistep procedure described above often entails the risk that the solutions are not sterile because they must be prepared by the user. In such cases, the safety of the patient is at risk because administration of unsterile solutions entails unforeseeable risks.

Because of the aforementioned disadvantages of the multistep procedure, it would be desirable if an active ingredient solution could be made available directly.

Earlier publications especially with regard to the stability of solutions and the solubility of ergot alkaloids have taught us that the disadvantages can be overcome to a great extent by using mixtures of water and alcohols as the solvent. To do so, the use of linear monofunctional alcohols and polyfunctional alcohols in amounts of 60.00% to 100.00% in particular has been described. Within the context of monofunctional alcohols, ethanol is used in particular. Representatives of polyfunctional alcohols that may be used include propylene glycol, glycerol or polyethylene glycol (GB 2 062 568 A, DE 27 35 587 A1, BE 881967 A, GB 1 539 083 A).

The following publications expand this information to mixtures consisting of water and exclusively polyfunctional alcohols with an alcohol content of 10.00% to 90.00%. In particular the use of 25.00% to 80.00% has proven suitable here (DD 43 402 A; EP 0 101 879 A2). This use is based on strictly oral therapy in these publications.

With regard to tolerability for the patient, there are in general problems with large amounts of cosolvents such as the alcohols mentioned above. Especially the use of high percentages of these compounds results in the solutions no longer meeting the requirements of the corresponding guidelines.

Furthermore, at higher concentrations of cosolvent, the viscosity of the solutions also increases. This may lead to problems in administration, e.g., when using a minipump or a micropump, because the solution is more difficult to pump.

Addition of corresponding alcohols in the literature has also been limited to use with naturally occurring ergot alkaloids and their 9,10-dihydro derivatives. Since these compounds do not have an 8α-amino group, they are significantly more stable than the ergot alkaloids with a corresponding amino group in α position in position 8 on the ring system. An amino group, optionally with additional substituents, has a greater tendency to instability. This pertains in particular to the diethylurea group —NHCON(C₂H₅)₂ as in the case of the lisuride, terguride, bromerguride or proterguride or their pharmaceutically tolerable salts and esters, for example. On the basis of this increased instability, the stabilization methods mentioned above are regarded as completely unsuitable for these compounds.

Therefore, the object of the present invention is to provide aqueous stabilized solutions of ergoline compounds of general formula I

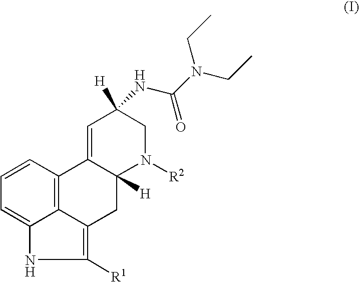

This object is achieved by a stabilized aqueous solution of an ergoline compound according to claim 1, which contains 0.05% to 90.00% of at least one oxygen-containing cosolvent. The percentage amounts in the following table are to be understood as mass per volume (m/v).

Additional preferred embodiments are derived from the dependent claims.

In other words, the object is achieved by a stabilized aqueous solution which contains an ergoline compound of formula I

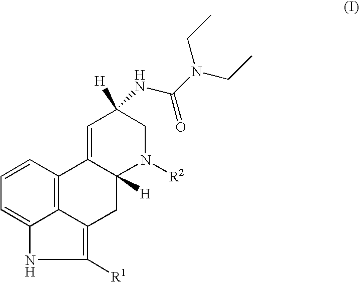

or its physiologically tolerable salt or derivative in which R¹ is a hydrogen atom or a halogen atom and R² as an alkyl group or an alkenyl group with 1 to 4 carbon atoms and [symbol] denotes a single bond or double bond and also 0.05% to 90.00% of at least one oxygen-containing cosolvent.

The at least one oxygen-containing cosolvent is preferably a polyvalent alcohol. It may preferably have 2 to 6 carbon atoms and at least two hydroxyl groups.

In a preferred embodiment, the polyvalent alcohol is selected from the group of 1,2-ethanediol (glycol), 1,2-propanediol (propylene glycol), 1,3-propanediol, 1,2,3-propanetriol (glycerol) or 1,3-butanediol.

The at least one oxygen-containing cosolvent is preferably also a polyethylene glycol with a molecular weight of 200-35,000 g/mol. It is especially preferable here for the at least one oxygen-containing cosolvent to be a polyethylene glycol with a molecular weight of 200-4,000 g/mol. The at least one oxygen-containing cosolvent is especially preferably a polyethylene glycol with a molecular weight of 400 g/mol.

The concentration of the at least one oxygen-containing cosolvent is preferably 0.05% to 20.00%. A concentration of the at least one oxygen-containing cosolvent of 0.50% to 9.50% is especially preferred.

In a preferred embodiment, the at least one oxygen-containing cosolvent is propylene glycol, and its concentration amounts to 0.05% to 9.50%.

In another preferred variant, the at least one oxygen-containing cosolvent is a nonionic detergent. It is preferably selected from the group of reaction products of polyethylene glycol, ethylene oxide or polyglycerol with fatty alcohols, alcohols, hydrogenated castor oil, fatty acids, hydroxy fatty acids or alkylphenols such as nonylphenol or derivatives thereof.

It is preferable here for the nonionic detergent to be selected from the group of reaction products of ethylene oxide and castor oil, the reaction products of hydrogenated castor oil and ethylene oxide or the polyethylene glycol 15 hydroxystearates, such as preferably Macrogol 15 hydroxystearate (Ph. Eur.) [European Pharmacopeia].

It is especially preferable for nonionic detergents from the group of reaction products of ethylene oxide and castor oil with a molar ratio of 20-60:1 or reaction products of hydrogenated castor oil and ethylene oxide with a molar ratio of 20-60:1 to be selected. The nonionic detergent is preferably selected from the group of reaction products of ethylene oxide and castor oil with a molar ratio of 30-60:1 or the reaction products of hydrogenated castor oil and ethylene oxide with a molar ratio of 30-60:1. Most highly preferably, the nonionic detergent is selected from the group of reaction products of ethylene oxide and castor oil with a molar ratio of 35:1 or the reaction products of hydrogenated castor oil and ethylene oxide with a molar ratio of 40:1 or 60:1.

In addition, it is preferable for the nonionic detergent to be selected from the group of polyoxysorbitan fatty acid esters, sorbitan fatty acid esters or polyoxyethylene-polyoxypropylenes.

The nonionic detergent is preferably present in a concentration of 0.05% to 90.00%. It is especially preferably present in a concentration of 0.20% to 20.00%. The nonionic detergent is most preferably present in a concentration of 0.20% to 10.00%.

The ergoline compound is preferably selected from the group of lisuride, terguride, proterguride and bromerguride. The ergoline compound is most preferably lisuride.

In a preferred embodiment, the ergoline compound is present in the form of its salt with sulfuric acid, sulfurous acid, phosphoric acid, phosphorous acid, nitric acid, nitrous acid, perchloric acid, hydrobromic acid, hydrochloric acid, formic acid, acetic acid, propionic acid, succinic acid, oxalic acid, gluconic acid (glyconic acid, dextronic acid), lactic acid, malic acid, tartaric acid, tartric acid (hydromalonic acid, hydroxypropane diacid), fumaric acid, citric acid, ascorbic acid, maleic acid, malonic acid, hydroxymaleic acid, pyruvic acid, phenylacetic acid, ortho-toluic acid, meta-toluic acid, para-toluic acid, benzoic acid, para-aminobenzoic acid, para-hydroxybenzoic acid, salicylic acid, para-aminosalicylic acid, methanesulfonic acid, ethanesulfonic acid, hydroxyethanesulfonic acid, ethylenesulfonic acid, para-toluenesulfonic acid, naphthylsulfonic acid, naphthylaminesulfonic acid, sulfanilic acid, camphorsulfonic acid, china acid (quinic acid), ortho-methylmandelic acid, hydrogenbenzenesulfonic acid, picric acid (2,4,6-trinitrophenol), adipic acid, D-(ortho-tolyl)tartaric acid or an amino acid.

Another preferred variant is implemented by the ergoline compound being present in the form of its salt with an amino acid from the group of methionine, tryptophan and arginine.

In addition, it is preferable for the ergoline compound to be present in the form of its salt with an acid-containing amino acid from the group of glutamic acid and aspartic acid.

An extremely preferred embodiment is implemented by the ergoline compound being present in the form of its salt with maleic acid.

The ergoline compound or its physiologically tolerable salt or derivative is preferably present in a concentration of 0.01 to 25.00 mg/mL. A concentration of the ergoline compound or its physiologically tolerable salt or derivative of 0.25 to 10.00 mg/mL is especially preferred. It is highly preferable for the ergoline compound or its physiologically tolerable salt or derivative to be present in a concentration of 0.50 to 3.00 mg/mL.

In addition, the stabilized aqueous solution may contain organic and/or inorganic compounds for adjusting the osmolarity in the case of a hypotonic solution and/or for adjusting the pH.

The stabilized solution is preferably stored in a prefilled syringe or in a capsule.

The invention also relates to the use of the stabilized aqueous solution for preparing an agent for parenteral treatment of neurodegenerative diseases or brain trauma. The stabilized aqueous solution includes an ergoline compound of formula I

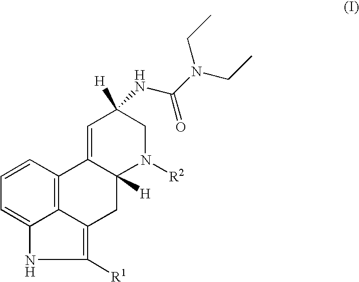

or its physiologically tolerable salt or derivative in which R¹ denotes a hydrogen atom or a halogen atom and R² denotes an alkyl group or an alkenyl group with 1 to 4 carbon atoms and [symbol] denotes a single bond or double bond which also contains 0.05% to 90.00% of at least one oxygen-containing cosolvent.

The parenteral treatment is preferably administered subcutaneously, intramuscularly, intravenously, transdermally or through a pump implanted in the bloodstream or in the tissue. With regard to pumps in general, many different pumps or micropumps may be used.

In one embodiment, the neurodegenerative diseases are Parkinson's disease or dystonias.

The brain trauma may be caused by a stroke or a traumatic brain injury.

It has surprisingly been found that the inventive ergoline compounds or their physiologically tolerable salts or derivatives may be dissolved in mixtures of water and at least one oxygen-containing cosolvent with a concentration of the cosolvent of 0.05% to 90.00%. It is also possible to use definitely lower concentrations than those described for stabilized ergot alkaloids in the state of the art. In particular, concentrations of 0.05% to 20.00%, especially preferably from 0.50% to 9.50% are possible here.

Since the at least one cosolvent surprisingly improves the solubility of the active ingredient, the volume to be administered to a patient each day in a parenteral dopaminergic therapy may be reduced significantly. The solubility of an inventive model compound has been improved by a factor of 1.5 by using an inventive cosolvent, in particular even by a factor of 2, where the concentration of the inventive cosolvent was less than 10% and the pH of the solution was neutral.

The resulting solutions have, contrary to all expectations, a stability of at least 6 months, in particular when using the alcohols listed above. The stabilization is even significantly improved with regard to increases in temperature. On the whole, in comparison with the unstabilized solution, an improved stability is observed even when using the nonionic detergents. When using the inventive alcohols, especially when using propylene glycol, the positive effect on solubility and stability of the invention ergoline compound is most noticeable. Especially with regard to the alcohols listed above, in particular propylene glycol, the result is a dual vantage on the whole: first the solubility of the respective active ingredient is increased and secondly the chemical stability of the respective active ingredient is surprisingly improved significantly.

In testing samples, it has been found, for example, that storage at 25° C. with light protection results in decomposition of 5% within a period of 180 days when not using a cosolvent according to this invention. With storage at 6-8° C., the decomposition amounted to 0.5% to 0.8%. In the case of storage at 40° C., up to 30% of the active ingredient had decomposed. In contrast with that, addition of an inventive cosolvent led to less than 0.5% decomposition with storage under refrigeration (6-8° C.) for 180 days in the absence of light. At room temperature (25° C.) an average decomposition of less than 0.5% was observed. In the case of storage at 40° C. in the presence of the inventive cosolvent, 89.8% of the active ingredient could still be detected, i.e., decomposition amounted to only approximately 10%.

Owing to the solutions stabilized according to this invention, it is now possible to supply patients directly with solutions. This is a significant improvement in comparison with the lyophilizates used in the past. At the same time, the problems with regard to sterility are also eliminated. Since the use of extremely low concentrations of the cosolvent was also surprisingly possible, parenteral administration is greatly facilitated. At low concentrations, the viscosity of the solutions can be kept in a range that is very suitable for handling. In addition, with the solutions stabilized according to this invention, it is possible to avoid exposing the patient to unnecessarily high doses of the alcohols over a long period of time.

For example, the estimated amount that is acceptable as a daily oral dose of propylene glycol is 25 mg/kg body weight (17th Report of the Joint FAO/WHO Expert Committee on Food Additives, 1974). Likewise, values for which daily amount is not acceptable for subcutaneous administration have also been published. Therefore, to reduce the safety risks for the patient, it is considered necessary to expose the patient to preferably only small quantities of the respective alcohol.

With the assumption of a daily dose of 2 mg lisuride hydrogen maleate, this yields a burden on the patient of 240 mg propylene glycol when using 8.00% propylene glycol and an assumed active ingredient concentration of 2 mg active ingredient per 3 mL solution. In the case of an average body weight of 70 kg, this dose thus amounts to less than 15% of the acceptable amount for oral administration. This can be optimized according to this invention by further increasing the active ingredient concentration and/or reducing the propylene glycol concentration.

The inventive stabilized aqueous solution may comprise organic and/or inorganic compounds, as mentioned above, to adjust the osmolarity in the case of a hypotonic solution and/or to adjust the pH.

By adjusting the pH, the solubility of the inventive ergoline compounds or their physiologically tolerable salts or derivatives can be increased. A reduction in pH leads to an increase in the amount of the ionized form of the inventive ergoline compound and thus to an improved solubility. For example, a reduction in pH from pH 7 to pH 6.2 with a representative inventive ergoline compound yields an improvement in solubility by a factor of approximately 5, and with a reduction in pH from 7 to 5.5 the solubility is even improved by a factor of approximately 20. However, the stability of the inventive ergoline compounds has an inverse relationship to the solubility. It has been observed that the stability becomes worse when the pH is reduced into the acidic range. On the whole, in adjusting the pH it has proven to be preferable to adjust a pH in the range of 4.00 to 8.00. A pH range from 4.50 to 7.50 is especially preferred, and a range of 5.00 to 7.00 is most especially preferred.

To adjust the pH, the stabilized aqueous solution may include a buffer system from the group of citrate buffer, carbonate buffer, phosphate buffer or maleate buffer. A citrate buffer is preferred as the buffer system.

The in-vivo tolerability of the inventive stabilized aqueous solution of an inventive ergoline compound or its physiologically tolerable salt or derivative can be optimized by adjusting the osmolarity. This is the case with an inventive solution in comparison with a solution that is hypotonic with blood. The term “hypotonic” as used here is understood to refer to a solution having a lower osmotic pressure than human blood. On the average, blood has an osmolarity (=Osmotic pressure) of 290 mosmol/L (milliosmol per liter). Then by adding physiologically tolerable excipients, the solution is preferably adjusted to an osmolarity of 250 to 350 mosmol/L, most preferably to an osmolarity of 270 to 320 mosmol/L. To adjust the osmolarity, sodium chloride is preferred. Sodium chloride, which is a physiologically tolerable excipient is preferably used to adjust a hypotonic solution that is isotonic with blood.

For structurally related active ingredients which are based on the same basic structure, similar physicochemical activities may be expected in vitro, in vivo and in clinical studies. The lisuride derivatives terguride and proterguride have alkyl substituents on the nitrogen (N6) of the basic structure and have a single bond between positions 9 and 10. In fact, these compounds yield a solubility in water comparable to that of lisuride. The solubility of the free proterguride amounts to 2.6 mg/100 mL, for example, in a phosphate-buffered solution (pH 7.4). The solubility of free lisuride may then be estimated at 2.2 mg/100 mL accordingly, but this value was calculated based on the solubility data of the respective active ingredients/active ingredient salts (I. Zimmermann (1983) International Journal of Pharmaceutics 13: 57-65).

The invention is illustrated below in greater detail on the basis of examples.

### EXAMPLES

### Example 1

### Stability of Lisuride in Buffered Aqueous Solutions

Solutions of lisuride hydrogen maleate with a concentration of 2 mg active ingredient per 3 mL solution were investigated with regard to their temperature-dependent stability. These solutions were buffered using a citrate buffer system, so that the pH value of the pure medium was adjusted to 5.1, 4.5 and 3.5. Aqueous solutions of citric acid monohydrate and trisodium citrate dihydrate (0.53 mM each) were prepared.

Suitable mixing of these solutions yielded aqueous buffer systems with a pH of 4.5 and/or 3.5. After 33.3 mg lisuride hydrogen maleate had been weighed out in a scaled 50 mL flask, 40 mL of the corresponding buffer medium was added. The flasks were agitated for several minutes and then exposed to ultrasound for several minutes until the active ingredient was completely dissolved. The solutions were cooled to room temperature and topped off to the mark with buffer medium. In conclusion, the flasks were again agitated for one minute.

In the case of the more strongly basic medium (pH 5.1), a supply solution was prepared, containing citric acid monohydrate (0.38 mM) and trisodium citrate dihydrate (0.68 mM). This was used to dissolve the active ingredient. In principle, double-distilled water was used to prepare the solutions.

The resulting solutions were stored under three different conditions: under refrigeration at 6 to 8° C., at room temperature (25° C.) and at an elevated temperature of 40° C. The solutions were stored in sealed glass ampoules which were wrapped with aluminum foil for protection from light. After suitable intervals of time, samples were analyzed by means of a specific reverse phase HPLC method with UV detection (running time 30 min, retention time of lisuride ˜13 min). It was observed that the decomposition of the active ingredient depends on the temperature and pH. The decomposition proceeded to a greater extent at high temperatures and at high concentrations of oxonium ions. In each of the cases, a decomposition of 5% was already discernible in storage at room temperature within a period of 180 days. In storage under refrigeration, the decomposition amounted to only approximately 0.7%. In the case of storage at 40° C., up to 30% of the active ingredient had decomposed.

All solutions that had been stored at 40° C. showed a significant yellow discoloration after only 30 days, turning brown after another 60 days.

TABLE 1
Purity data for lisuride hydrogen maleate
dissolved in aqueous citrate-buffered solutions.

| Refrigeration | | | Room temperature | | | Elevated temperature | | |
|---|---|---|---|---|---|---|---|---|
| (6-8° C.) | | | (25° C.) | | | (40° C.) | | |
| | Average | Area | t | Average | Area | t | Average | Area |
| t [d] | area | SD | [d] | area | SD | [d] | area | SD |

pH 5.1

| | | | | | | | | |
|---|---|---|---|---|---|---|---|---|
| 0 | 100.00 | | 0 | 100.00 | | 0 | 100.00 | |
| 7 | 99.90 | 0.05 | 7 | 99.79 | 0.04 | 7 | 98.86 | 0.06 |
| 30 | 99.72 | 0.03 | 30 | 99.36 | 0.03 | 30 | 94.71 | 0.26 |
| 90 | 99.59 | 0.02 | 90 | 98.64 | 0.14 | 90 | 90.99 | 0.37 |
| 180 | 99.29 | 0.04 | 180 | 95.45 | 1.17 | 180 | 87.98 | 0.61 |

pH 4.5

| | | | | | | | | |
|---|---|---|---|---|---|---|---|---|
| 0 | 99.79 | | 0 | 99.79 | | 0 | 99.79 | |
| 7 | 99.88 | 0.03 | 7 | 99.84 | 0.01 | 7 | 98.82 | 0.12 |
| 30 | 99.73 | 0.01 | 30 | 99.26 | 0.06 | 30 | 94.96 | 0.09 |
| 90 | 99.75 | 0.11 | 90 | 98.14 | 0.28 | 90 | 90.39 | 0.30 |
| 180 | 99.11 | 0.13 | 180 | 95.90 | 1.19 | 180 | 87.71 | 0.19 |

pH 3.5

| | | | | | | | | |
|---|---|---|---|---|---|---|---|---|
| 0 | 99.87 | | 0 | 99.87 | | 0 | 99.87 | |
| 7 | 99.92 | 0.01 | 7 | 99.89 | 0.01 | 7 | 98.89 | 0.02 |
| 30 | 99.80 | 0.02 | 30 | 99.37 | 0.07 | 30 | 90.41 | 0.12 |
| 90 | 99.57 | 0.07 | 90 | 98.70 | 0.13 | 90 | 73.37 | 1.15 |
| 180 | 99.22 | 0.15 | 180 | 94.14 | 1.90 | 180 | 69.91 | 0.89 |

The data are given in percent of the lisuride peak area relative to the total peak area of the chromatogram.
“SD” stands for standard deviation.

### Example 2

### Addition of 9% Propylene Glycol

An aqueous solution of lisuride hydrogen maleate which additionally contained 9% propylene glycol was investigated. The active ingredient concentration was adjusted to 2 mg per 3 mL. No buffer system was added. The remaining procedure included weighing 33.3 mg of the active ingredient into a scaled 50 mL flask, then adding the propylene glycol (4.5 g) and also adding 40 mL water. The flask was agitated for several minutes and then exposed to ultrasound until the active ingredient was completely dissolved. The flask was cooled to room temperature and topped off with double-distilled water up to the mark. In conclusion, to ensure homogeneity, agitation was continued for another minute.

The solution was poured into transparent glass ampoules with a volume of 1 mL. These ampoules were sealed airtight and wrapped with aluminum foil to protect them from light. The ampoules were each stored at 6-8° C., 25° C. and 40° C. HPLC analysis revealed that these solutions were significantly more stable than the solutions prepared without the addition of propylene glycol (see Example 1).

After storage under refrigeration for 180 days, approximately 0.4% of the active ingredient had decomposed. The samples stored at room temperature also revealed a remaining active ingredient content of 98.4% in comparison with the initial concentration. In the case of storage at 40° C., only approximately 10% of the active ingredient had decomposed.

In addition, the discoloration of the solutions occurred with a time lag in comparison with the solutions from Example 1. A slight yellow discoloration was observed only after a storage time of 90 days at an elevated temperature.

Another advantage of using propylene glycol as the low-dose additive (less than 10%) was the improved solubility of the active ingredient (lisuride hydrogen maleate). Accordingly, the volume to be administered to a patient each day can be greatly reduced after optimizing the drug concentration in a parenteral dopaminergic treatment of neurodegenerative diseases.

TABLE 2
Purity data on lisuride hydrogen maleate dissolved in
double-distilled water with the addition of 9% propylene glycol.

| Refrigeration | | | Room temperature | | | Elevated temperature | | |
|---|---|---|---|---|---|---|---|---|
| (6-8° C.) | | | (25° C.) | | | (40° C.) | | |
| | Average | Area | t | Average | Area | t | Average | Area |
| t [d] | area | SD | [d] | area | SD | [d] | area | SD |
| 0 | 99.80 | | 0 | 99.80 | | 0 | 99.80 | |
| 7 | 99.79 | 0.09 | 7 | 99.79 | 0.01 | 7 | 99.33 | 0.06 |
| 30 | 99.67 | 0.01 | 30 | 99.46 | 0.02 | 30 | 97.59 | 0.18 |
| 90 | 99.52 | 0.05 | 90 | 99.15 | 0.02 | 90 | 94.06 | 0.93 |
| 180 | 99.36 | 0.08 | 180 | 98.35 | 0.34 | 180 | 89.76 | 0.50 |

The data are given in percent of the lisuride peak area relative to the total peak area of the chromatogram.
“SD” stands for standard deviation.

### Example 3

### Addition of Cremophor ELP

An aqueous lisuride hydrogen maleate solution containing various additives was investigated with regard to its improved stability. The additives used were propylene glycol and Cremophor ELP in concentrations between 1.00% and 10.00%.

The solutions were prepared by analogy with the procedure used in examples 1 and 2.

The solutions were stored in the dark in glass containers at 60° C. for one week.

It was found that propylene glycol would lead to a significantly improved stability of the active ingredient in comparison with Cremophor ELP. The remaining active ingredient content when using propylene glycol was 92.5%. In contrast with that, under the same conditions when using Cremophor ELP, then degradation of the active ingredient amounted to 14.3-21.3% (1% and 10% Cremophor ELP, respectively).

This also points to the better suitability of propylene glycol in particular in comparison with other additives because on the whole this yields a dual advantage. First, the solubility of the respective active ingredient is increased and secondly, the chemical stability of the respective active ingredient is surprisingly also improved.

1. A stabilized aqueous solution of an ergoline compound of formula I 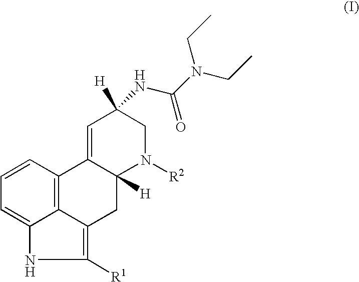
or its physiologically tolerable salt or derivative, in which R¹ denotes a hydrogen atom or a halogen atom and R² denotes an alkyl group or alkenyl group with 1 to 4 carbon atoms and [symbol] denotes a single bond or a double bond, characterized in that the aqueous solution also comprises 0.05% to 90.00% (m/v) of at least one oxygen-containing cosolvent.

2. The stabilized aqueous solution according to claim 1, characterized in that the at least one oxygen-containing cosolvent is a polyvalent alcohol.

3. The stabilized aqueous solution according to claim 1, characterized in that the polyvalent alcohol has 2 to 6 carbon atoms and at least two hydroxyl groups.

4. The stabilized aqueous solution according to claim 1, characterized in that the polyvalent alcohol is selected from the group including 1,2-ethanediol, 1,2-propanediol, 1,3-propanediol, 1,2,3-propanetriol or 1,3-butanediol.

5. The stabilized aqueous solution according to claim 1, characterized in that the at least one oxygen-containing cosolvent in a polyethylene glycol with a molecular weight of 200-35,000 g/mol.

6. The stabilized aqueous solution according to claim 1, characterized in that the at least one oxygen-containing cosolvent is a polyethylene glycol with a molecular weight of 200-4,000 g/mol.

7. The stabilized aqueous solution according to claim 1, characterized in that the at least one oxygen-containing cosolvent is a polyethylene glycol with a molecular weight of 400 g/mol.

8. The stabilized aqueous solution according to claim 1, characterized in that the concentration of the at least one oxygen-containing cosolvent is 0.05% to 20.00%.

9. The stabilized aqueous solution according to claim 1, characterized in that the concentration of the at least one oxygen-containing cosolvent is 0.50% to 9.50%.

10. The stabilized aqueous solution according to claim 1, characterized in that the at least one oxygen-containing cosolvent is propylene glycol and its concentration is 0.05% to 9.50%.

11. The stabilized aqueous solution according to claim 1, characterized in that the at least one oxygen-containing cosolvent is a nonionic detergent.

12. The stabilized aqueous solution according to claim 1, characterized in that the nonionic detergent is selected from the group of reaction products of polyethylene glycol, ethylene oxide or polyglycerol with fatty alcohols, alcohols, hydrogenated castor oil, fatty acids, hydroxy fatty acids or alkylphenols such as nonylphenol or derivatives thereof.

13. The stabilized aqueous solution according to claim 1, characterized in that the nonionic detergent is selected from the group of reaction products of ethylene oxide and castor oil, the reaction products of hydrogenated castor oil and ethylene oxide or polyethylene glycol 15 hydroxystearate.

14. The stabilized aqueous solution according to claim 1, characterized in that the polyethylene glycol 15 hydroxystearate is Macrogol 15 hydroxystearate (Ph. Eur.).

15. The stabilized aqueous solution according to claim 1, characterized in that the nonionic detergent is selected from the group of reaction products of ethylene oxide and castor oil, having a molar ratio of 20-60:1 or the reaction products of hydrogenated castor oil and ethylene oxide having a molar ratio of 20-60:1.

16. The stabilized aqueous solution according to claim 1, characterized in that the nonionic detergent is selected from the group of reaction products of ethylene oxide and castor oil, with a molar ratio of 30-60:1 or the reaction products of hydrogenated castor oil and ethylene oxide with a molar ratio of 30-60:1.

17. The stabilized aqueous solution according to claim 1, characterized in that the nonionic detergent is selected from the group of reaction products of ethylene oxide and castor oil, with a molar ratio of 35:1 or the reaction products of hydrogenated castor oil and ethylene oxide with a molar ratio of 40:1 or 60:1.

18. The stabilized aqueous solution according to claim 1, characterized in that the nonionic detergent is selected from the group of polyoxysorbitan fatty acid esters, sorbitan fatty acid esters or polyoxyethylene polyoxypropylenes.

19. The stabilized aqueous solution according to claim 1, characterized in that the nonionic detergent is present in a concentration of 0.05% to 90.00%.

20. The stabilized aqueous solution according to claim 1, characterized in that the nonionic detergent is present in a concentration of 0.20% to 20.00%.

21. The stabilized aqueous solution according to claim 1, characterized in that the nonionic detergent is present in a concentration of 0.2% to 10.00%.

22. The stabilized aqueous solution according to claim 1, characterized in that the ergoline compound is selected from the group of lisuride, terguride, proterguride and bromerguride.

23. The stabilized aqueous solution according to claim 1, characterized in that the ergoline compound is lisuride.

24. The stabilized aqueous solution according to claim 1, characterized in that the ergoline compound is present in the form of its salt with sulfuric acid, sulfurous acid, phosphoric acid, phosphorous acid, nitric acid, nitrous acid, perchloric acid, hydrobromic acid, hydrochloric acid, formic acid, acetic acid, propionic acid, succinic acid, oxalic acid, gluconic acid, lactic acid, malic acid, tartaric acid, tartronic acid, fumaric acid, citric acid, ascorbic acid, maleic acid, malonic acid, hydroxymaleic acid, pyruvic acid, phenylacetic acid, ortho-toluic acid, metatoluic acid, para-toluic acid, benzoic acid, para-aminobenzoic acid, para-hydroxybenzoic acid, salicylic acid, para-aminosalicylic acid, methanesulfonic acid, ethanesulfonic acid, hydroxyethanesulfonic acid, ethylenesulfonic acid, para-toluenesulfonic acid, naphthylsulfonic acid, naphthylaminesulfonic acid, sulfanilic acid, camphorsulfonic acid, quinic acid, orthomethymandelic acid, hydrogenbenzenesulfonic acid, picric acid, adipic acid, D-(ortho-toly)tartaric acid or an amino acid.

25. The stabilized aqueous solution according to claim 1, characterized in that the ergoline compound is present in the form of its salt with amino acid from the group of methionine, tryptophan and arginine.

26. The stabilized aqueous solution according to claim 1, characterized in that the ergoline compound is present in the form of its salt with an acid-containing amino acid from the group of glutamic acid and aspartic acid.

27. The stabilized aqueous solution according to claim 1, characterized in that the ergoline compound is present in the form of its salt with maleic acid.

28. The stabilized aqueous solution according to claim 1, characterized in that the ergoline compound or its physiologically tolerable salt or derivative is present in a concentration of 0.01 to 25.00 mg/mL.

29. The stabilized aqueous solution according to claim 1, characterized in that the ergoline compound or its physiologically tolerable salt or derivative is present in a concentration of 0.25 to 10.00 mg/mL.

30. The stabilized aqueous solution according to claim 1, characterized in that the ergoline compound or its physiologically tolerable salt or derivative is present in a concentration of 0.50 to 3.00 mg/mL.

31. The stabilized aqueous solution according to claim 1, characterized in that the solution also comprises organic and/or inorganic compounds for adjusting the osmolarity in the case of a hypnotic solution and/or for adjusting the pH.

32. The stabilized aqueous solution according to claim 1, characterized in that the solution comprises sodium chloride to adjust the osmolarity.

33. The stabilized aqueous solution according to claim 1, characterized in that it has an osmolarity of 250 to 350 mosmol/L.

34. The stabilized aqueous solution according to claim 1, characterized in that it has an osmolarity of 270 to 320 mosmol/L.

35. The stabilized aqueous solution according to claim 1, characterized in that the solution comprises a buffer system from the group of citrate buffer, carbonate buffer, phosphate buffer or maleate buffer to adjust the pH.

36. The stabilized aqueous solution according to claim 1, characterized in that the solution comprises a citrate buffer as the buffer system.

37. The stabilized aqueous solution according to claim 1, characterized in that it has a pH in the range of 4.00 to 8.00.

38. The stabilized aqueous solution according to claim 1, characterized in that it has a pH in the range of 4.50 to 7.50.

39. The stabilized aqueous solution according to claim 1, characterized in that it has a pH in the range of 5.00 to 7.00.

40. A use of a stabilized aqueous solution according to claim 1 for producing an agent for parenteral treatment of neurodegenerative diseases or brain trauma, characterized in that the stabilized aqueous solution is an ergoline compound of formula I 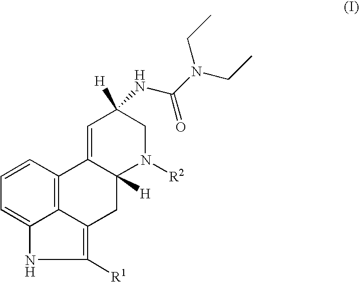
or its physiologically tolerable salt or derivative, where R¹ denotes a hydrogen atom or a halogen atom and R² denotes an alkyl group or an alkenyl group with 1 to 4 carbon atoms and [symbol] denotes a single bond or a double bond, characterized in that the aqueous solution also contains 0.05% to 90.00% (m/V) of at least one oxygen-containing cosolvent.

41. The use of a stabilized aqueous solution according to claim 1, for production of an agent for parenteral treatment of neurodegenerative diseases or brain trauma, characterized in that the parenteral treatment is administered subcutaneously, intramuscularly, intravenously, transdermally or through a pump implanted in the blood stream or the tissue.

42. The use of a stabilized aqueous solution according to claim 1, characterized in that the neurodegenerative diseases include Parkinson's disease or dystonias.

43. The use of a stabilized aqueous solution according to claim 1, characterized in that the brain trauma is caused by a stroke or a traumatic brain injury.
